(19) United States
(12) Patent Application Publication
(10) Pub. No.: US 2006/0142689 A1
(43) Pub. Date: Jun. 29, 2006

(54) Systems, methods and compositions for cleaning wounds

(76) Inventors: Suraj Puri, Los Altos, CA (US); George Plaut, San Jose, CA (US)

Correspondence Address: DECHERT LLP, P.O. BOX 10004, PALO ALTO, CA, 94303, US

(21) Appl. No.: 11/339,250

(22) Filed: Jan. 24, 2006

Related U.S. Application Data
(63) Continuation-in-part of application No. 10/965,697, filed on Oct. 13, 2004.

(51) Int. Cl. A61B 17/20

(52) U.S. Cl. 604/22

(57) ABSTRACT

Systems, methods and compositions for effectively cleaning wounds are described. According to the present invention, wound care is promoted by the use of a charged solution and particularly in the presence of sonic energy. Inventive methods for producing a solution which promotes removal of microbes, comprising charging a solution that contains at least a solute selected to promote removal of microbes, to produce a charged solution. As a result of charging, at least a portion of the solute is present as clusters in the charged solution. Alternatively, the solute is selected to promote formation of solute clusters, which effectively remove microbes found on or inside the wound. Inventive systems for applying the charged cleaning solution includes: (i) a housing capable of holding a pressurized-charged solution; and (ii) a sonic chamber capable of introducing sonic frequency into the pressurized-charged solution to form a sonically activated charged solution.

### CROSS-REFERENCE TO RELATED APPLICATIONS

This application is a continuation-in-part of U.S. patent application Ser. No. 10/965,697, filed Oct. 13, 2004, entitled “SYSTEM, METHODS AND COMPOSITIONS FOR PROMOTING ORAL HYGIENE” (Attorney Docket No. 374308-00201 (349966)).

### FIELD OF THE INVENTION

The present invention relates to systems and methods for effectively removing different types of microbes. More particularly, the present invention relates to compositions, systems and methods for effectively removing microbes that form in or on wounds.

### BACKGROUND OF THE INVENTION

Wound cleaning has consistently strived to devise new designs, methods and compositions for killing various microbes that grow on different tissue present in or on the wound. To date, the basic process for killing microbes has not been able to effectively kill all or substantially all of the microbes that grow on or inside the wound. The conventional wound cleaning approach relies on identifying the many different types microbes that grow in our inside the wounds, then developing genomic data on each of the identified microbes and then formulating a solution which destroys each of the different microbes. Unfortunately, the conventional approach of wound cleaning suffers from several drawbacks. For example, the process of identifying microbes, developing genomic data and formulating the appropriate solution to destroy the microbes is time-consuming and very expensive. As another example, each of these steps have to be performed accurately such that many different microbes such that each of the many different types of microbes are effectively destroyed. This problem is further exacerbated by the fact that these microbes often mutate as the conventionally wound cleaning process is being implemented. As a result, a resulting the convention wound cleaning solution, which may have been initially formed to kill a specific microbe, may no longer kill the microbe it was designed for because that microbe has mutated.

What is therefore needed is an improved system, process or composition for effectively cleaning wounds, without suffering from the drawbacks encountered by the conventional cleaning processes.

### SUMMARY OF THE INVENTION

To achieve the foregoing, the present invention provides systems, methods and compositions for effectively cleaning wounds by removing the many different microbes that grow on or inside the wound, as opposed to destroying these microbes. By removing, and not killing, such microbes, the present invention circumvents the drawbacks associated with identifying microbes, developing genomic data and formulating the appropriate destroying solution, all of which are encountered by the conventional cleaning solutions. Specifically, the present invention provides systems, methods and compositions for producing a solution which effectively removes a substantial amount of each of the different types of microbes found on or inside a wound. Based on these inventions, the present invention also provides inventive methods of facilitating wound care.

In one aspect the present invention provides a method for producing a solution which promotes removal of microbes. The method includes charging a solution, which contains at least a solute selected to promote removal of microbes, to produce a charged solution, wherein as a result of the charging, at least a portion of the solute is present as clusters in the charged solution.

In another aspect, the present invention provides a system for making a cleaning solution which promotes removal of microbes. The system includes: (1) a charging chamber for holding a solution, which contains at least a solute selected to promote formation of solutes in cluster form; and (2) a first acoustic energy source capable of vibrating the solution in the charging chamber to produce a charged solution, wherein at least a portion of the solute is present as clusters in the charged solution.

In yet another aspect, the present invention provides a method for facilitating removal of a bio-film or a microbe. The method includes applying a charged solution to a bio-film or a microbe and providing sonic energy to the charged solution during the step of applying a charged solution to the bio-film or a microbe.

In yet another aspect, the present invention provides a composition of a solution for promoting removal of bio-film or a microbe. The composition includes a solvent and a solute that is selected to promote formation of solute clusters, wherein at least a portion of at least one molecule of the cluster is surrounded by a plurality of solvent molecules.

In yet another aspect, the present invention provides an assembly for applying a charged solution to a bio-film or a microbe. The assembly includes a housing capable of holding a pressurized charged solution, and a sonic chamber capable of introducing sonic frequency into the pressurized charged solution to form a sonically activated charged solution.

In a preferred embodiment of the inventive assemblies, there is also a cap which is capable of attaching to the assembly, such that the cap is communicatively coupled to the sonic chamber to receive the sonically activated charged solution and the cap is designed for dispensing the sonically activated charged solution to wound tissue. In certain embodiments of the present invention, the sonic chamber is activated when a low voltage signal is supplied to the sonic chamber. In other alternative embodiments of the present invention, the inventive assemblies include a delivery mechanism capable of pressurizing a charged solution and delivering the pressurized charged solution to the housing. Such a delivery mechanism may include a pump, which pressurizes the charged solution and delivers the pressurized charged solution to the housing. Such inventive assemblies may further include a reservoir (e.g., an intravenous bag) of charged solution that is communicatively coupled to the delivery mechanism to convey the solution from the reservoir to the delivery mechanism. In preferred embodiments, the sonic chamber includes any one of a pulsating device or piezo-electric device.

In yet another aspect, the present invention provides a method for producing a solution which promotes removal of microbes. The method includes charging a solution, which contains at least a solute selected to promote formation of clusters, to produce a charged solution, wherein as a result of the charging, produces at least a portion of the solute in cluster form surrounded by solvent in the charged solution.

In yet another aspect, the present invention provides a method for removing microbes. The method includes: (1) charging a solution, which contains at least a solute selected to promote formation of clusters, to produce a charged solution, wherein as a result of the charging, at least a portion of the solute is present as clusters in the charged solution; and (2) applying the charged solution to the microbes to remove a substantial portion of the microbes.

In preferred embodiments of the present invention, the step of applying includes applying the charged solution to the microbes in the presence of a sonic energy and microbes include those microbes that are capable of forming a bio-film.

These and other features of the present invention will be further described in the following detailed description of the invention with reference to the associated drawings.

### BRIEF DESCRIPTION OF THE FIGURES

FIG. 1 is a schematic of a system, according to one embodiment of the present invention, for producing a solution that is for effectively cleaning wounds according to one embodiment of the present invention.

FIG. 2A shows the random distribution of the solute particles in an uncharged solution.

FIG. 2B shows the solute particles arranged in cluster form in a charged solution, according to one embodiment of the present invention.

FIG. 3 shows one embodiment of a delivery system of the present invention that is used to deliver the charged solution to a wound.

### DETAILED DESCRIPTION OF THE INVENTION

The present invention provides systems, methods and compositions for effectively cleaning wounds. Conventional wisdom dictates that for effectively cleaning wounds, high enough concentrations of a solute, which is typically an active cleaning ingredient in the cleaning solution, should be used. The solute in the conventional cleaning solution is specifically designed to attack and destroy a particular type of microbe that is found on or inside a wound. According to such wisdom, it is believed that high concentrations of such solute particles react with the boundary of the different microbes to kill the microbes. Predicated on this belief, conventional wisdom teaches away from using cleaning solutions with relatively low solute concentrations or low concentration of the active ingredient in the cleaning process.

In the present invention, however, relatively low solute concentrations of the solute are preferred because they promote charging of a solution. For more information on the concept of charging, reference can be made to U.S. patent application Ser. No. 10/886,785, filed on Jul. 7, 2004 and entitled “SYSTEMS AND METHODS FOR CHARGING A CLEANING SOLUTION USED FOR CLEANING INTEGRATED CIRCUIT SUBSTRATES,” which is incorporated herein by reference for all purposes. It is believed that in a charged solution, solute particles are arranged as clusters. It is also believed that these solute clusters trap detached microbes for effective removal from on or inside the wound. As a result, the present invention focuses on forming solute clusters for effective wound cleaning. Notably, the inventive cleaning systems, methods and compositions described herein not only provide a way to clean wound using relatively low solute concentrations, which are deemed ineffective by conventional cleaning techniques, rather such low solute concentrations represent preferred embodiments of the present invention.

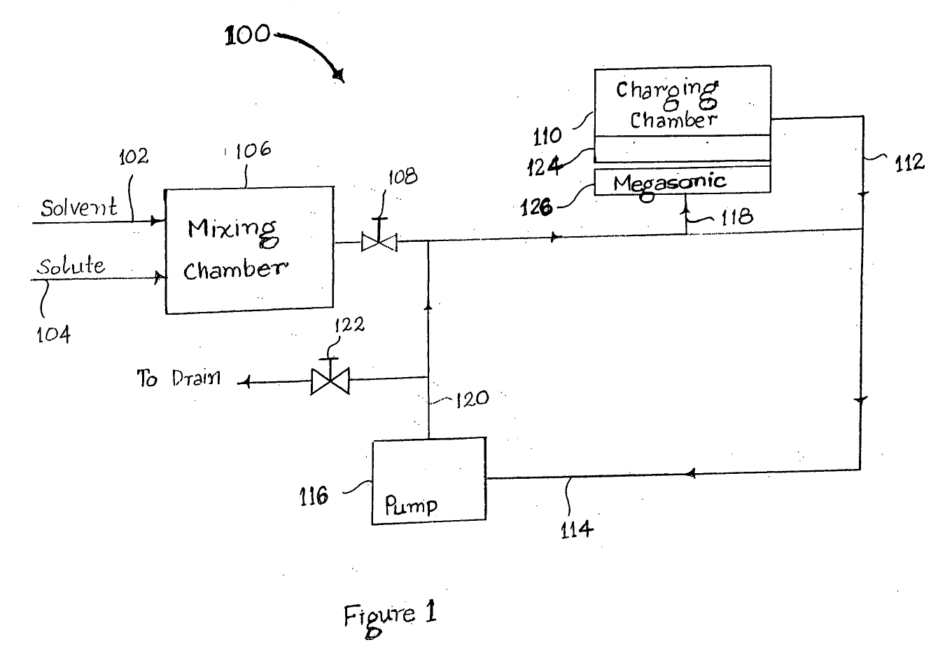

FIG. 1 shows a charging system 100, according to one embodiment of the present invention, for producing a charged solution that facilitates wound cleaning. System 100 is shown to have two chambers—a mixing chamber 106 for mixing a solute stream 104 and a solvent stream 102 to form a solution, and a charging chamber 110 for charging the solution formed in mixing chamber 106. A valve 108 is activated to convey the contents of mixing chamber 106 to charging chamber 110. Charging chamber 110 comes equipped with a first acoustic energy source 126 and a coupling chamber 124 for coupling the continuous energy transmission into the charging chamber.

A first connection 118 between mixing chamber 106 and charging chamber 110 allows the uncharged solution to be conveyed from chamber 106 to chamber 110 for charging. After the solution inside chamber 110 is charged, a pump 116 conveys the charged solution from that chamber via a second connection 112, an optional third connection 114, an optional fourth connection 120 to drain or, in the alternative, for further recharging back to chamber 110. In one embodiment, in system 100, a valve 122 and a fourth connection 120 facilitates conveying the charged solution to drain where it is recovered for use. Typically, drain here represents a location where the charged solution may be retrieved and then stored in a vial or container and can be subsequently used by a user to promote wound care. If the solution in chamber 110 is not charged to the desired extent, the same pathway (i.e., connections 112, 114 and 120) direct the charged solution to combine with the uncharged solution exiting from chamber 106. The combination of these two streams is introduced into chamber 110 for further charging.

Mixing chamber 106 may be any equipment known in the art that can controllably combine a flow of at least one liquid with a flow of at least one gas. Charging chamber 110 can be made of any material known to be a good transmitter of acoustic energy. This chambers is preferably made from quartz. Although FIG. 1 describes two chambers as two separate vessels, it is possible that the two chambers could be incorporated into a single vessel. The preferred embodiment, however, is to have two separate vessels as shown in FIG. 1.

Acoustic energy source 126 can be any source that supplies megasonic energy and the like. Charging chamber 110 is ideally placed above acoustic energy source 126 and coupling chamber 124. Use of megasonic energy is, however, preferred because it is more effective at removing smaller particles from the tissue surface. Although the megasonic device used in system 100 can have outputs as high as 5 Watts/cm² and higher, it is preferable to use an output of 3 Watts/cm² and lower. Suitable equipment for generating megasonic energy is commercially available from a variety of vendors. Such equipment should, however, preferably include a generator and a series special transducers or the like. By way example, megasonic devices, which are commercially available from Kaijo Corporation of Japan, Prosys of Cambell, Calif. and PCT Systems, Inc. of Fremont, Calif. work well.

A typical process in system 100 for producing the wound cleaning solution begins when a solvent stream 102, typically a stream of deionized water, flows into mixing chamber 106. Similarly, a solute stream 104 enters the same chamber 106 so mixing may commence. Solute is typically any solute that facilitates removal of a particulatar microbe found on or inside the wound. Alternatively, the solute can also be any chemical species that promotes formation of clusters in a solution. In certain embodiments of the present invention, the solute is sodium chloride or ammonium hydroxide. In a preferred embodiment, the solute is typically ammonia gas, which allows using ammonium hydroxide in extremely low concentrations. In such preferred embodiments, before mixing ammonia gas with deionized water, it is filtered to bring its purity to 99.99999%. Those skilled in the art will recognize that depending on the types of microbes to be removed from the tissue surface, other types of solutes, different from sodium chloride ammonium hydroxide, may be used. By way of example, a solution includes other chemicals, such as 03, H₂O₂, and NH₄OH. According to the present invention, such solutes promote charging a solution, i.e., forming solute particles in cluster form in the solution. These solutes are usually mixed with deionized water to form a solution which is subsequently used for wound cleaning. In cluster form, one or more solute particles are surrounded by numerous molecules of deionized water. The concentration of the mixing solution is generally between about 1×10⁻⁶ and about 1×10⁻⁹, and preferably between about 3×10⁻⁵ and about 1×10⁻⁶.

By activating valve 108, a solution formed in mixing chamber 106 that is in its uncharged state, is conveyed to charging chamber 110 for charging. An acoustic energy source 126, preferably a megasonic device, through a coupling chamber 124 supplies sufficient energy to charge the fluid to create a coherent liquid inside charging chamber 110. As a result, inside chamber 110, a relatively dilute solution is transformed from an uncharged state to a charged state by the aid of a megasonic device.

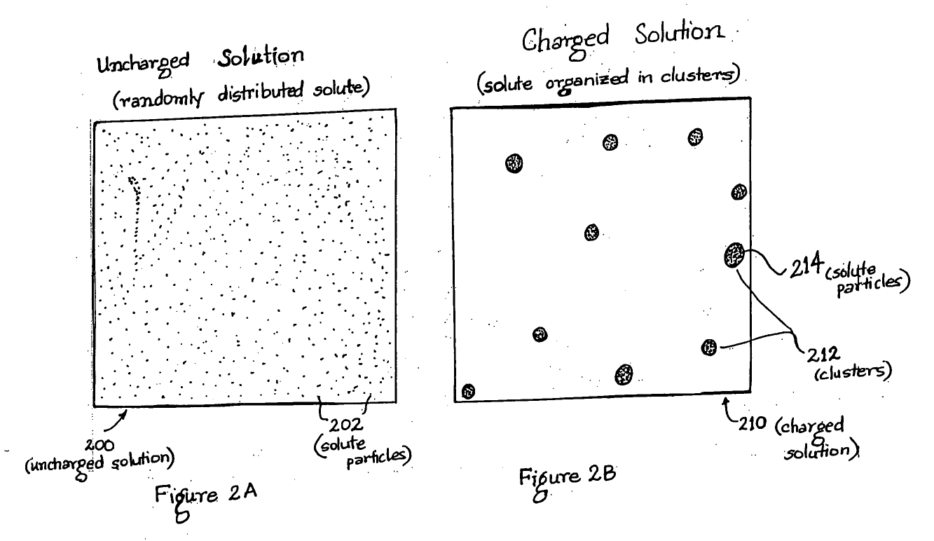

FIGS. 2A and 2B illustrate the difference in solute particle distribution from a solution's uncharged state to a charged state. In FIG. 2A, which shows an uncharged solution 200, solute particles 202 are randomly distributed throughout solution 200. In FIG. 2B, which shows a charged solution 210, solute particles 214 are organized in solution 210 as clusters 212. In a preferred embodiment of the present invention, an average number of molecules of solute 214 in clusters 212 is between about 100 and about 200 molecules per cluster. Those skilled in the art will recognize that in each of solutions 200 and 210, the solute particles are dissolved in a solvent, which is not shown in FIGS. 2A and 2B to simplify illustration and facilitate discussion. Furthermore, clusters 212 need not have a circular shape with a smooth exterior, as shown in FIG. 2B. Rather, clusters 212 are generally of any irregular shape.

The charged solution exiting charging chamber 110 has solute present in the solvent in a volumetric ratio that is between about 5×10⁻⁵:1 and about 1×10⁻²⁴:1, preferably between about 1×10⁻⁶:1 and about 1×10⁻²⁴:1 and more preferably between about 1×10⁻⁸:1 and about 1×10⁻²⁴:1. It is important to note, however, that after the first charging action in chamber 110, the exiting solution sometimes may not be charged to the desired extent.

To this end, in preferred embodiments as shown in FIG. 1, system 100 is designed to charge to a greater extent a charged solution stream flowing out of charging chamber 110. Second, third and fourth connections 112, 114 and 120 define a pathway that facilitates, among other things, diluting this charged solution using more solvent from solvent stream 102 and conveying the diluted solution to charging chamber 110 for further charging. In such dilute charged solution, solute is present in a solvent in a volumetric ratio that is between about 5×10⁻⁵:1 and about 1×10⁻²⁴:1. This preferred embodiment, provides the flexibility of using small amounts of charged solution residing in charging chamber 110 and further diluting it to produce a more effectively charged solution.

The charged solution obtained by activating valve 122 of FIG. 1 has a composition for promoting wound care. This composition primarily includes a solute, which selected to promote cleaning of tissue present on or inside the wound. Alternatively, in the present invention, the solute can also be selected because it promotes producing a solution in a charged state. In certain embodiments of the present invention, an active ingredient responsible for detaching microbes from the tissue surface is typically present in the cleaning solution as the solute. For example, the solute is one member selected from the group consisting of antibacterial agents or disinfectants. In the present invention, when the cleaning solution is in a charged state, at least a portion of the solute is present in cluster form in the solution. Cleaning a wound using solute clusters represents a marked improvement over the conventional cleaning compositions. In conventional cleaning compositions, the solute, which is typically present in relatively high concentrations, may be able to kill the microbe from the wound tissue by a chemical reaction at the tissue surface or temporarily detach the microbe from the wound tissue by mechanical action of the flowing solution. However, the detached microbe may not have sufficient energy to be completely removed from the wound. The microbes, as a result, return and adhere to the wound tissue surface. While wishing not to be bound by theory, in the composition, according to the present invention, the solute clusters trap the detached microbes and effectively prevent them from returning to the wound tissue surface. Accompanying mechanical action during wound cleaning and particle debridement, such as a pulsating action, remove the trapped microbes or particles from the wound surface. A subsequent rinse cycle ensures that these trapped microbes or particles are completely removed from the wound surface. By way of example, an aspirated suction may be applied to remove the trapped microbes or particles in the charged solution.

To use the inventive cleaning compositions for removing microbes or particles from wound tissue requires, among other things, applying the above-described charged solution to the tissue present in wound, and providing sonic energy to the charged solution contained on or inside the wound. The present invention relies upon high pH of the charged near-zero solute dilutions in the cleaning solution to capture and remove the microbes or particles, whose detachment is aided by using sonic energy. In preferred embodiments of the present invention, sonic energy, in addition to the presence of charged solution, is applied to the wound tissue to facilitate the removal of the contaminants from the tissue surface.

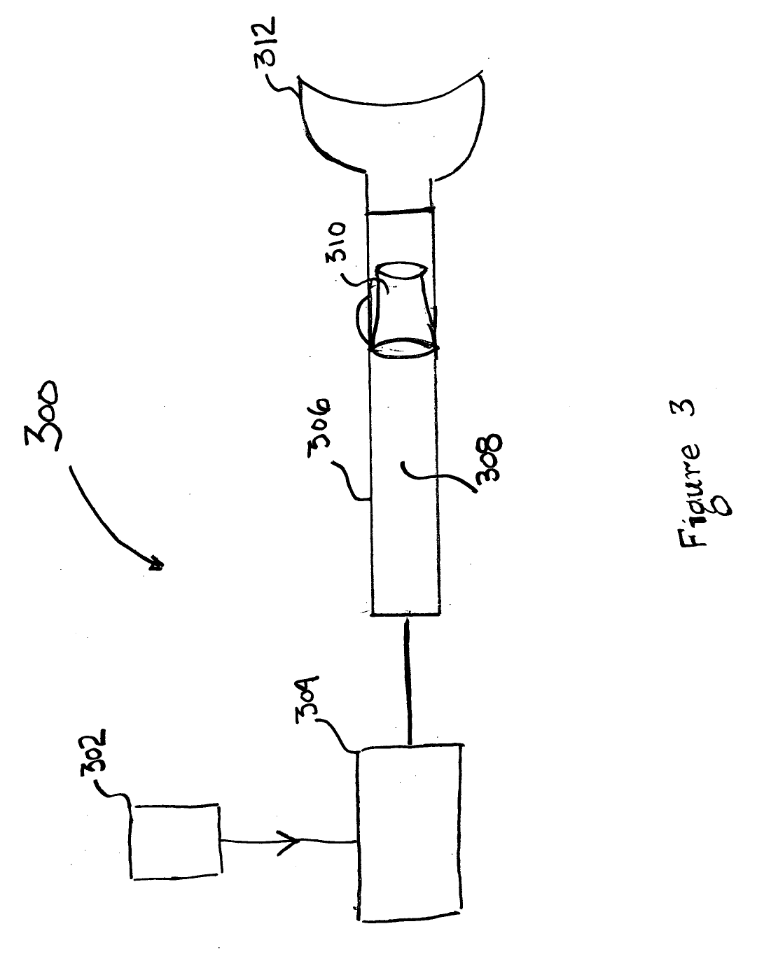

In accordance with one embodiment of the present invention, FIG. 3 shows a delivery system 300 for applying the charged solution to the wound tissue. FIG. 3 shows a delivery mechanism 304 for pressurizing a charged solution contained in a charged solution reservoir 302 to a wand 306. Delivery mechanism 304 connects to wand 306 by any technique well known to those skilled in the art. By way of example, delivery mechanism 304 includes a flexible line that provides the pressurized solution to wand 306. Wand 306 includes a housing 308 designed for holding a pressurized charged solution, a sonic chamber 310 and a protective cap 312.

Charged solution reservoir 302 can be a vial or a container in which the drain from valve 122 of FIG. 1 empties. In other words, the charged solution produced by a charging process described with respect to FIG. 1 is stored in charged solution 302. In one embodiment of the present invention, charged solution is communicatively coupled via a flexible line to convey the charged solution to delivery mechanism 304. Delivery mechanism 304 may come equipped with a pump, which both pressurizes the charged solution received from reservoir 302 to produce a pressurized charged solution and deliver that pressurized charged solution to wand 306 via a flexible line, for example.

Wand 306 is preferably a cylindrical shaped body with an inlet and an outlet. The inlet of wand 306 is designed to communicatively couple a housing 308 inside wand 306 and delivery mechanism 304. Housing 308 is, in turn, design to hold the pressurized charged solution received from delivery mechanism 304. Sonic chamber 310 disposed between housing 308 and outlet of wand 306 preferably includes a device that introduces sonic energy into the pressurized charged solution. In preferred embodiments of the present invention, the sonic device in sonic chamber 310 is either a pulsating device or a piezo-electric device to effectively sonically charge the pressurized charged solution. A protective cap 312 is communicatively coupled to sonic chamber 310 such that the sonically-pressurized charged solution is received by cap 312, which ultimately dispenses this solution to the wound.

During a typical application using assembly 300 of FIG. 3, a charged solution from reservoir 302 is conveyed to a delivery mechanism 304, where it is pumped and pressurized for delivery to wand 306. The pressurized charged solution through an inlet of 306 enters housing 308. When activated, the pressurized charged solution flows past sonic chamber 310, where sonic energy is introduced into the pressurized charged solution to produce a sonically-pressurized charged solution. The sonically-pressurized charged solution is applied to a wound through protective cap 312. Those skilled in the art will recognize that a sonic device, e.g., a pulsating device or piezo-electric device, inside sonic chamber 310 may be activated by low voltage source. Furthermore, those skilled in the art will also recognize that wand 306 may also include provisions to supply the required low voltage signal to the sonic device inside sonic chamber 310. Further still, those skilled in the art will recognize that these embodiments also apply to lavage therapies.

The inventive wound application assemblies or lavage therapies described herein represent a marked improvement over the conventional assemblies. Specifically, the inventive assemblies supply sonic energy to a charged solution or the tissue at the point of use. Such application of sonic energy is not realized by conventional assemblies because according to conventional wisdom, it is not relevant to provide such sonic energy either to the conventional cleaning solution or to the tissue that is undergoing cleaning.

Although certain examples have been described in terms of cleaning wounds, those skilled in the art will recognize that the inventive systems, methods and compositions described herein can be used for cleaning internal wounds as well as external wounds.

What is claimed is:

1. A method for producing a solution which promotes removal of microbes, comprising charging a solution, which contains at least a solute selected to promote removal of microbes, to produce a charged solution, wherein as a result of said charging, at least a portion of said solute is present as clusters in said charged solution.

2. The method of claim 1, wherein said charging is carried out by vibrating said solution to produce at least said portion of said solute in cluster form.

3. The method of claim 2, wherein said vibrating is caused by sonic energy.

4. The method of claim 3, wherein said vibrating is caused by ultrasonic energy or megasonic energy.

5. The method of claim 4, wherein said microbes is present on tissue that is wounded.

6. The method of claim 1, wherein said solute is any one member selected from the group consisting of any organic or inorganic substances that can be used as a solute.

7. The method of claim 6, wherein said solute is any one member selected from the group consisting of sodium chloride and ammonium hydroxide.

8. The method of claim 1, wherein an average number of molecules of said solute in said clusters is between about 100 and about 200 molecules per cluster.

9. The method of claim 1, wherein said charging further comprising diluting said solution such that said solute is present in a solvent in a volumetric ratio that is between about 5×10⁻⁵:1 and about 1×10⁻²⁴:1.

10. The method of claim 9, wherein said solute is present in said solvent in a volumetric ratio that is between about 1×10⁻⁶:1 and about 1×10⁻²⁴:1.

11. The method of claim 10, wherein said solute is present in said solvent in a volumetric ratio that is between about 1×10⁻⁸:1 and about 1×10⁻²⁴:1.

12. The method of claim 1, further comprising:
diluting said charged solution to produce a cleaning solution; and
using said cleaning solution for promoting removal of said microbes.

13. The method of claim 1, wherein said charging includes applying acoustic energy for producing said clusters.

14. The method of claim 1, further comprising mixing a solvent and said solute to produce said solution before said charging said solution.

15. The method of claim 14, wherein said solvent is deionized water.

16. The method of claim 14, wherein said mixing produces said solution having said solute present in said solvent in a volumetric ratio that is between about 3×10⁻⁵:1 and about 1×10⁻²⁴:1.

17. A system for making a cleaning solution which promotes removal of microbes, comprising:
a charging chamber for holding a solution, which contains at least a solute selected to promote formation of solutes in cluster form; and
a first acoustic energy source capable of vibrating said solution in said charging chamber to produce a charged solution, wherein at least a portion of said solute is present as clusters in said charged solution.

18. The system of claim 17, further comprising a mixing chamber for mixing a solvent and said solute to produce said solution.

19. The system of claim 18, further comprising a connection which allows communication between said charging chamber and an exit stream of said mixing chamber such that said solvent and solute combine with the contents of said charging chamber to produce an effective cleaning solution.

20. A method for facilitating removal of a bio-film or a microbe, comprising:
applying a charged solution to a bio-film or a microbe; and
providing sonic energy to said charged solution during said applying.

21. The method of claim 20, further comprising supplying sonic energy to said bio-film or a microbe.

22. The method of claim 21, wherein said providing is carried out by a piezo-electrical mechanism.

23. The method of claim 20, wherein said providing is carried out by using an acoustic energy producing mechanism.

24. A composition of a solution for promoting removal of bio-film or a microbe, comprising:
a solvent; and
a solute selected to promote formation of solute clusters, wherein at least a portion of at least one molecule of said cluster is surrounded by a plurality of solvent molecules.

25. The composition of claim 24, wherein said solute and solvent are present in a volumetric ratio that is between about 1:1 and about 1×10⁻²⁴:1.

26. The composition of claim 24, wherein said solute is sodium chloride and ammonium hydroxide.

27. The composition of claim 24, wherein said solvent is deionized water.

28. The composition of claim 24, wherein said solute and solvent are present in a volumetric ratio that is between about 1×10⁻⁵:1 and about 1×10⁻²⁴:1.

29. The composition of claim 24, wherein said solute and solvent are present in a volumetric ratio that is between about 1×10⁻⁷:1 and about 1×10⁻²⁴:1.

30. The composition of claim 24, wherein said solute and solvent are present in a volumetric ratio that is between about 1×10⁴:1 and about 1×10⁻⁸:1.

31. An assembly for applying a charged solution to a bio-film or a microbe, comprising:
a housing capable of holding a pressurized charged solution; and
a sonic chamber capable of introducing sonic frequency into said pressurized charged solution to form a sonically activated charged solution.

32. The assembly of claim 31, further comprising a cap being capable of attaching to said assembly, such that said cap is communicatively coupled to said sonic chamber to receive said sonically activated charged solution and said cap being designed for dispensing said sonically activated charged solution to wound tissue.

33. The assembly of claim 31, wherein said sonic chamber is activated when a low voltage signal is supplied to said sonic chamber.

34. The assembly of claim 31, further comprising a delivery mechanism capable of pressurizing a charged solution and delivering said pressurized charged solution to said housing.

35. The assembly of claim 34, wherein said delivery mechanism includes a pump which pressurizes said charged solution and delivers said pressurized charged solution to said housing.

36. The assembly of claim 34, further comprising a reservoir of charged solution that is communicatively coupled to said delivery mechanism to convey said solution from said reservoir to said delivery mechanism.

37. The assembly of claim 31, wherein said reservoir is an intravenous bag.

38. The assembly of claim 31, wherein said sonic chamber includes any one of a pulsating device or piezo-electric device.

39. A method for producing a solution which promotes removal of microbes, comprising charging a solution, which contains at least a solute selected to promote formation of clusters, to produce a charged solution, wherein as a result of said charging, at least a portion of said solute is present as clusters in said charged solution.

40. A method for removing microbes, comprising:
charging a solution, which contains at least a solute selected to promote formation of clusters, to produce a charged solution, wherein as a result of said charging, at least a portion of said solute is present as clusters in said charged solution; and
applying said charged solution to said microbes to remove a substantial portion of said microbes.

41. The method of claim 40, wherein said applying includes applying said charged solution to said microbes in the presence of a sonic energy.

42. The method of claim 40, wherein said microbes include microbes that are capable of forming a bio-film.
